(19) United States
(12) Patent Application Publication
(10) Pub. No.: US 2004/0043974 A1
(43) Pub. Date: Mar. 4, 2004

(54) Oxidation process for preparing the intermediate 6.alpha.,9.alpha.-defluoro-11.beta.,17.alpha.-dihydroxy-16.alpha.-methyl-androst-1,4-dien-3-one 17.beta.-carboxylic acid

(76) Inventors: Frederick David Albinson, Stevenage (GB); Steven John Coote, Stevenage (GB); John Malcolm Robinson, Stevenage (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/333,537

(22) PCT Filed: Jul. 20, 2001
(86) PCT No.: PCT/GB01/03289

(30) Foreign Application Priority Data
Jul. 21, 2000 (GB) 0017988.7

(51) Int. Cl. A61K 31/56; C07J 3/00

(52) U.S. Cl. 514/179; 552/610

(57) ABSTRACT

The present invention relates to a process for the preparation of a compound of formula (I) which comprises the following step:

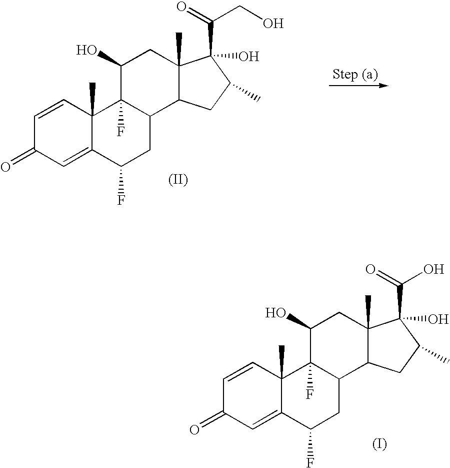

wherein step (a) comprises oxidation of a solution containing the compound of formula (II) wherein the solution is formed in a mixture of water and tetrahydrofuran.

The present invention relates to a novel process for the synthesis of a known intermediate, useful in the preparation of anti-inflammatory steroids. There is also provided a new physical form of the intermediate which has improved handling properties

6α, 9α-difluoro-11β-hydroxy-16α-methyl-17α-propionyloxy-3-oxoandrosta-1, 4-diene-17β-carbothioic acid, S-fluoromethyl ester (Formula A) was first described as an anti-inflammatory steroid by U.S. Pat. No. 4,335,121. This compound is also known by the generic name of fluticasone propionate and has since become widely known as a highly effective steroid in the treatment of inflammatory diseases, such as asthma and chronic obstructive pulmonary disease (COPD).

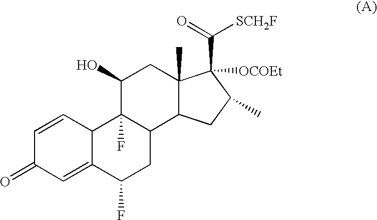

Additionally, 6α, 9α-difluoro-11β-hydroxy-16α-methyl-3-oxo-17α-propionyloxy-androsta-1,4-diene-17β-carbothioic acid S-(2-oxo-tetrahydro-furan-3-yl) ester (Formula B) was described in WO 97/24365 as a class of hydrolysable steroids with lactone derivatives. This compound possesses useful anti-inflammatory activity whilst having little or no systemic activity.

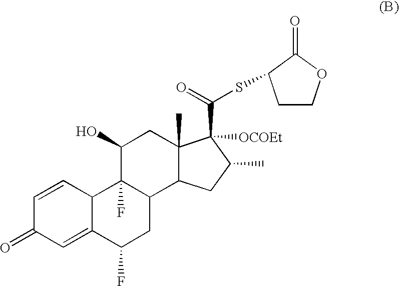

Currently there is considerable interest in compounds of formula (A) and (B) as anti-inflammatory and anti-allergic compounds in the treatment of asthma and other inflammatory diseases.

U.S. Pat. No. 4,335,121 and WO 97/24365 describe preparations of fluticasone propionate and 6α, 9α-difluoro-11β-hydroxy-16α-methyl-3-oxo-17α-propionyloxy-androsta-1,4-diene-17β-carbothioic acid S-(2-oxo-tetrahydro-furan-3-yl) ester, respectively which utilise a common starting material, namely 6α, 9α-difluoro-11β-hydroxy-16α-methyl-3-oxo-17α-hydroxy-androsta-1,4-diene-17β-carboxylic acid (Formula I). However, this hydroxy acid androstane derivative compound is an extremely costly starting material for preparing quantities of steroids of formulae (A) and (B).

Thus, according to a first aspect of the present invention we provide a novel process for the preparation of a compound of formula (I) which comprises the following step:

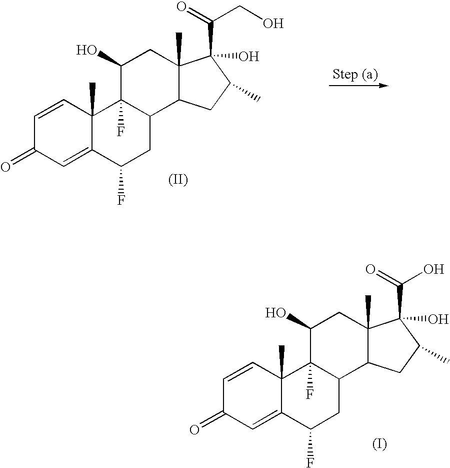

wherein step (a) comprises oxidation of a solution containing the compound of formula (II).

Preferably, step (a) will be performed in the presence of a solvent comprising methanol, water, tetrahydrofuran, dioxan or diethylene glygol dimethylether. For example, so as to enhance yield and throughput, preferred solvents are methanol, water or tetrahydrofuran, and more preferably are water or tetrahydrofuran, especially water and tetrahydrofuran as solvent. Dioxan and diethylene glygol dimethylether are also preferred solvents which may optionally (and preferably) be employed together with water.

Preferably, the solvent will be present in an amount of between 3 and 10 vol relative to the amount of the starting material (1 wt.), more preferably between 4 and 6 vol., especially 5 vol.

If desired, the solution containing the compound of formula (II) may be cooled prior to oxidation eg. to a temperature less than approximately 10° C.

Preferably the oxidising agent is present in an amount of 1-9 molar equivalents relative to the amount of the starting material. For example, when a 50% w/w aqueous solution of periodic acid is employed, the oxidising agent may be present in an amount of between 1.1 and 10 wt. relative to the amount of the starting material (1 wt.), more preferably between 1.1 and 3 wt., especially 1.3 wt.

Preferably, the oxidation step will comprise the use of a chemical oxidising agent. More preferably, the oxidising agent will be periodic acid or iodic acid or a salt thereof. Most preferably, the oxidising agent will be periodic acid or sodium periodate, especially periodic acid. Alternatively (or in addition), it will also be appreciated that the oxidation step may comprise any suitable oxidation reaction, eg. one which utilises air and/or oxygen. When the oxidation reaction utilises air and/or oxygen, the solvent used in said reaction will preferably be methanol.

Preferably, step (a) will involve incubating the reagents at room temperature or a little warmer, say around 25° C. eg for 2 hours.

The compound of formula (I) may be isolated by crystallisation from the reaction mixture by addition of an anti-solvent. A suitable anti-solvent for compound of formula (I) is water. Surprisingly we have discovered that it is highly desirable to control the conditions under which the compound of formula (I) is precipitated by addition of anti-solvent eg water. When the crystallisation is performed using chilled water (eg water/ice mixture at a temperature of 0-5 ° C.) although better anti-solvent properties may be expected we have found that the crystalline product produced is very voluminous, resembles a soft gel and is very difficult to filter. Without being limited by theory we believe that this low density product contains a large amount of solvated solvent within the crystal lattice. By contrast when conditions of around 10° C. or higher are used (eg around ambient temperature) a granular product of a sand like consistency which is very easily filtered is produced. Under these conditions, crystallisation typically commences after around 1 hour and is typically completed within a few hours (eg 2 hours). Without being limited by theory we believe that this granular product contains little or no of solvated solvent within the crystal lattice.

As a further aspect of the invention we provide a process for preparing a compound of formula (I) which comprises the steps of

(a) oxidation of a solution containing the compound of formula (II) followed by

(b) precipitation of the compound of formula (I) in crystalline form from the reaction mixture by addition of an anti-solvent under temperature conditions of around 10° C. or higher.

Preferably the anti-solvent is water. Preferably the temperature of step (b) is ambient temperature (eg around 18-22° C.) or higher (eg up to 40° C.).

We also provide as an aspect of the invention the compound of formula (I) in the form of a granular solid which is readily filterable obtainable by a process comprising:

(a) oxidising of a solution of a compound of formula (II) in water/tetrahydrofuran wth periodic acid in water at a temperature of around 20-30° C.; followed by

(b) precipitating the compound of formula (I) by addition of water at a temperature of around 22° C.

As a further aspect of the present invention we provide a novel process for the preparation of a compound of formula (A) which comprises the following step:

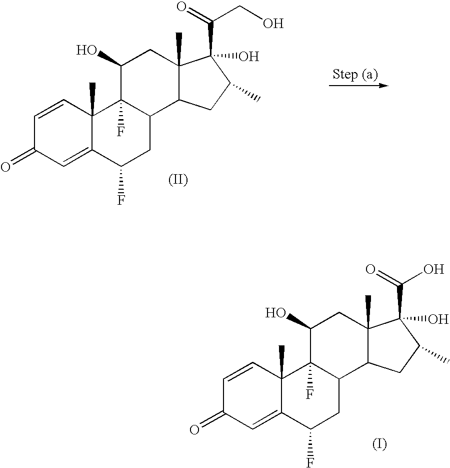

wherein step (a) comprises oxidation of a solution containing the compound of formula (II).

A preferred embodiment of the present invention is wherein the process for the preparation of a compound of formula (A) comprises the following steps:

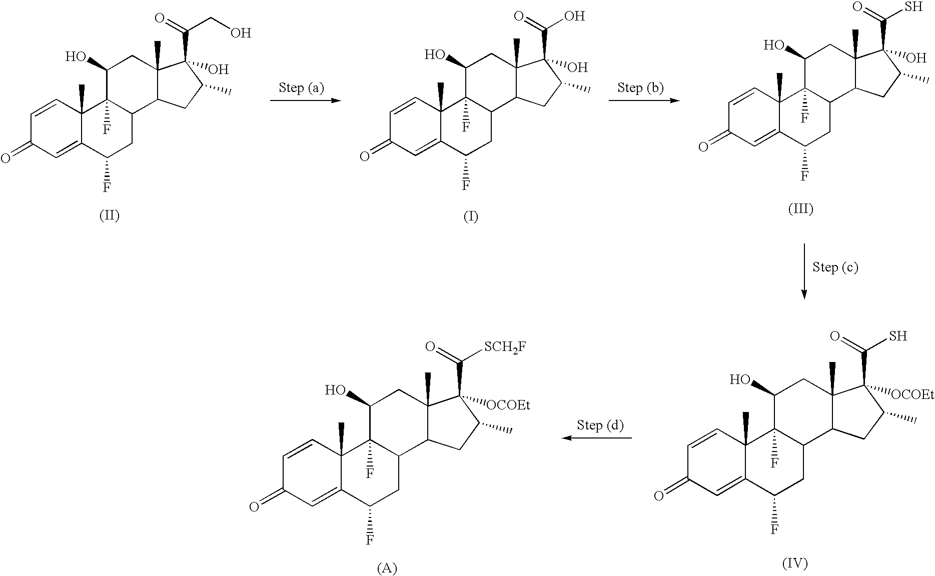

wherein step (a) comprises oxidation of a solution containing the compound of formula (II).

Step (b) will typically comprise the addition of a reagent suitable for converting a carboxylic acid to a carbothioic acid eg. using hydrogen sulphide gas together with a suitable coupling agent eg. carbonyldiimidazole (CDI) in the presence of a suitable solvent eg. dimethylformamide.

Step (c) typically comprises the addition of a reagent suitable for performing the esterification to the ethyl ester eg. propionyl chloride in the presence of suitable solvents eg. diethylamine, triethylamine, dichloroniethane and acetone. Step (d) typically comprises the addition of a reagent suitable for performing alkylation eg. either by direct conversion by addition of a haloalkyl compound or via an iodinated intermediate compound.

As a further aspect of the present invention we also provide a novel process for the preparation of a compound of formula (B) which comprises the following step:

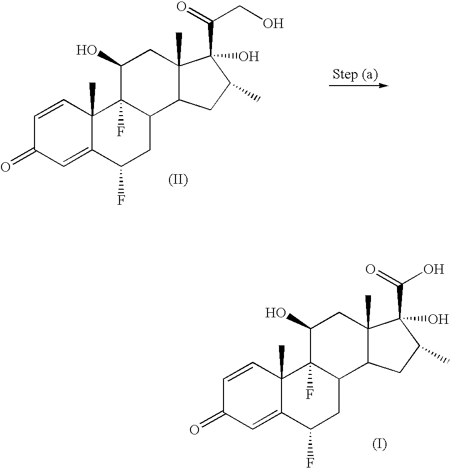

wherein step (a) comprises oxidation of a solution containing the compound of formula (II).

A preferred embodiment of the present invention is wherein the process for the preparation of a compound of formula (B) comprises the following steps:

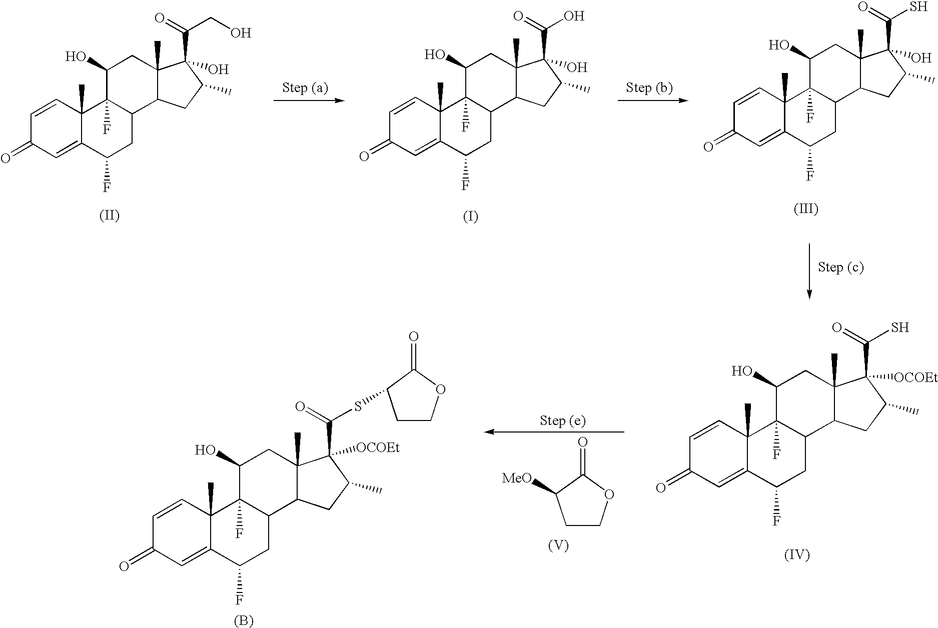

wherein step (a) comprises oxidation of a solution containing the compound of formula (II).

Typical conditions for step (b) and (c) are as previously described. Typically, step (e) will comprise reagents suitable to effect the coupling of compounds of formula (IV) with compounds of formula (V) eg. in the presence of a suitable solvent eg. dimethylformamide together with a suitable base eg. 2,4,6-collidine, pyridine or caesium carbonate. Compounds of formula (B) may then be optionally purified by suitable recrystallisation processes eg. recrystallisation from suitable solvents eg. isopropanol, diethylketone or methyl isobutyl ketone.

Compounds of formula (V) may be prepared according to the following process:

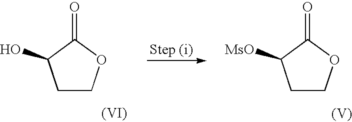

wherein step (i) typically comprises the addition of a suitable reagent eg. methanesulphonyl chloride in the presence of solvents eg. triethylamine, dimethylaminopyridine and ethyl acetate.

In step (e), analogues of compounds of formula (V) in which the MsO-group is replaced with another leaving group may also be employed.

It will be understood that this novel process makes use of an alternative starting material, flumethasone (formula (II)). Surprisingly, it has been shown that the use of such a starting material in the process for preparing fluticasone propionate and 6α, 9α-difluoro-11β-hydroxy-16α-methyl-3-oxo-17α-propionyloxy-androsta-1,4-diene-17β-carbothioic acid S-(2-oxo-tetrahydro-furan-3-yl) ester would substantially reduce production costs for these steroids.

As a further aspect of the present invention we also provide the use of a compound of formula (II):

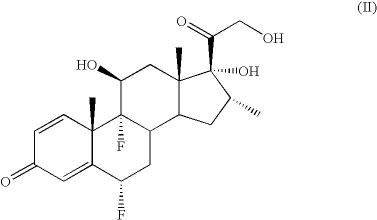

in the preparation of a compound of formula (I):

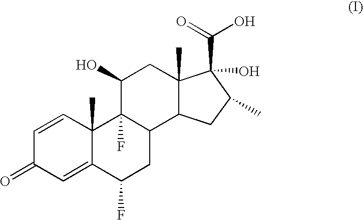

As a further aspect of the present invention we also provide the use of a compound of formula (II):

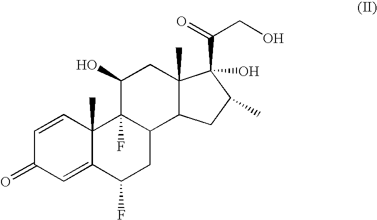

in the preparation of a compound of formula (A):

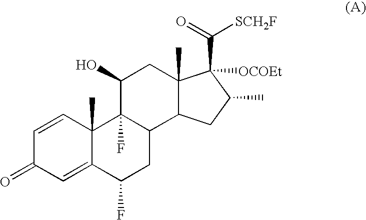

As a further aspect of the present invention we also provide the use of a compound of formula (II):

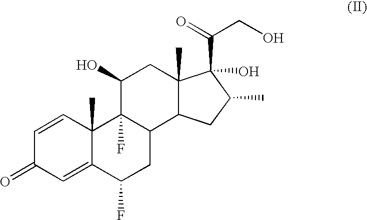

in the preparation of a compound of formula (B):

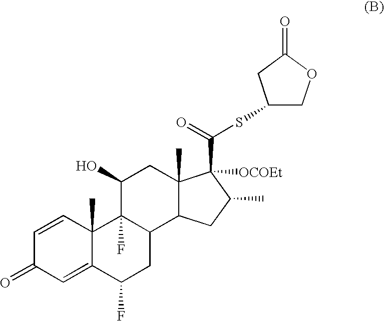

The present invention is illustrated by the following Examples:

Intermediate 1: 6α, 9α-difluoro-11β, 17α-dihydroxy-16α-methyl-3-oxoandrosta-1,4-diene-17β-carbothioic acid

A solution of Example 1 (12.0 g) in dry dimethylformamide (250 ml) was stirred and treated with N,N′-carbonyldiimidazole (9.94 g) under nitrogen at room temperature. After 4 hours, hydrogen sulphide was passed through the solution for 0.5 hours. The reaction mixture was poured into 2M hydrochloric acid (500 ml) containing ice (approximately 250 g). The resulting precipitate was collected, washed with water and dried in vacuo to give the title compound as a white solid (11.47 g), m.p. 230-232° C., [α]_{D}+94° C. (c 0.91).

Intermediate 2: 6α, 9α-difluoro-11β-hydroxy-16α-methyl-3-oxo-17α-propionyloxy-androsta-1,4-diene-17β-carbothioic acid

A solution of Intermediate 1 (5.0 g) and triethylamine (6.15 ml) in dichloromethane (140 ml) was cooled with ice-salt and treated dropwise with propionyl chloride (4.74 ml). The reaction mixture was stirred further at approximately 0° C. for 0.75 hours then washed successively with 2M sodium carbonate, water, 2M hydrochloric acid, water and brine. After being dried, solvent was removed to give a white solid (6.35 g). This was redissolved in acetone (120 ml) and diethylamine (12.5 ml): after being stirred at room temperature for 1 hour the volume was reduced to approximately 75 ml. The solution was poured into 2M hydrochloric acid (200 ml) containing ice (approximately 300 g) and the resulting precipitate was collected, washed with water and dried in vacuo to a white solid (5.17 g) m.p. 152-155° C. Recrystallisation of a portion (400 mg) from ethyl acetate gave the analytically pure title compound as colourless crystals (290 mg), m.p. 161 -164° C., [α]_{D}−27° C. (c 0.95).

Intermediate 3: Methansulfonic acid 2-oxo-tetrahydro-furan-3R-yl ester

Triethylamine (1.5 vol, 1.1 eq) and 4-N,N-dimethylaminopyridine (0.012 wt, 0.01 eq) in ethylacetate (2 vol) is added to a stirred solution of (R)-(+)-α-hydroxy-γ-butyrolactone (1 wt, 1 eq) in ethyl acetate (12 vol) under nitrogen at 20° C.±3° C.

The solution is cooled to below 10° C. and methanesulphonyl chloride (0.79 vol, 1.05 eq) is cautiously added to the reaction mixture over a period of at least 15 minutes at a rate sufficient to maintain the reaction temperature below 35° C. After complete addition, the reaction mixture is cooled to 20° C.±3° C. and stirred for up to 7 h at 20° C.±3° C. under nitrogen, monitoring for completion by TLC (EtOAc, cyclohexane; 1:1; staining solution: 3 g KMnO₄, 20 g K₂CO₃, 0.5 g NaOH, and 300 ml water) or GC. Upon completion, the reaction mixture is treated with 1M HCl (3 vol) and stirred until all solids have dissolved. The phases are separated and the organic phase is washed with further 1M HCl (3 vol). The phases are separated and the organic phase is distilled under reduced pressure to approximately 4 vol using a rotary evaporator. The organic solution is heated to 40-50° C. and treated with cyclohexane (12 vol). The mixture is cooled to below 15° C. and aged at 10-15° C. for at least 15 minutes. The mixture is filtered, the collected solid is washed with cyclohexane (2×3 vol) and dried under vacuum at 30-35° C. to yield the title compound as a white solid. Expected yield: 150% w/w, 85% theory.

### EXAMPLE 1

6α, 9α-difluoro-11β-hydroxy-16α-methyl-3-oxo-17α-hydroxy-androsta-1,4-diene-17β-carboxylic acid

A suspension of flumethasone (40 g) in tetrahydrofuran (199 ml) was treated with lab grade water (13.2 ml) and stirred at 20° C. until a clear solution was achieved (approximately 2 minutes). The solution was cooled to less than 10° C. and an aqueous solution of periodic acid (99% purity, 33.32 g (1.5 mole equivs) in water (68 ml)) was added dropwise over a period of 6 minutes. The clear solution was allowed to warm to ambient (approximately 20° C.) and stirred at ambient for 2 hours and 5 minutes. HPLC analysis at 90 minutes showed 97.4area % title compound present in the reaction mixture. Water (1000 ml) was added dropwise over a period of 15 minutes causing crystallisation/precipitation of the product. After complete addition, the mixture was externally cooled and aged at approximately 10° C. for 100 minutes and the product filtered off. The bed was washed with water (3×140 ml) and dried at 70° C. (house vacuum) for 26 hours and 40 minutes leaving the title compound as a white granular solid (37.98 g, 98.3% th).

### EXAMPLE 1A

6α, 9α-difluoro-11β-hydroxy-16α-methyl-3-oxo-17α-hydroxy-androsta-1,4-diene-17β-carboxylic acid

A suspension of flumethasone (5 g, 12.2 mmol) in dioxan (22 ml) and lab grade water (3 ml) was treated with an aqueous solution of periodic acid (50% w/w purity, 6.65 g, 14.6 mmol (1.2 mole equivs)) over a period of 45 minutes keeping the temperature in the range 25-30° C. The suspension was stirred at ambient for 2 hours. HPLC analysis at near 2 hours showed 98.1area % title compound present in the reaction mixture. Water (70 ml) was added dropwise over a period of 45 minutes. After complete addition, the mixture was stirred 20° C. for 1 hour and the product filtered off. The bed was washed with water (2×15 ml) and dried at 60° C. (house vacuum) for 18 hours leaving the title compound as a white granular solid (4.65 g, 96.3% th).

### EXAMPLE 1B

6α, 9α-difluoro-11β-hydroxy-16α-methyl-3-oxo-17α-hydroxy-androsta-1,4-diene-17β-carboxylic acid

A suspension of flumethasone (5 g, 12.2 mmol) in diethylene glygol dimethyl ether (22 ml) and lab grade water (4.4 ml) was treated with an aqueous solution of periodic acid (50% w/w purity, 6.65 g, 14.6 mmol (1.2 mole equivs)) over a period of 45 minutes keeping the temperature in the range 25-30° C. The suspension was stirred at ambient for 5 hours. HPLC analysis at near 5 hours showed 95.8area % title compound present in the reaction mixture. Water (70 ml) was added dropwise over a period of 45 minutes. After complete addition, the mixture was stirred at 20° C. for 1 hour and the product filtered off. The bed was washed with water (2×15 ml) and dried at 60° C. (house vacuum) for 72 hours leaving the title compound as a white granular solid (4.66 g, 96.5% th).

### EXAMPLE 1C

6α, 9α-difluoro-11β-hydroxy-16α-methyl-3-oxo-17α-hydroxy-androsta-1,4-diene-17β-carboxylic acid

A suspension of flumethasone (5 g, 12.2 mmol) in tetrahydrofuran (22 ml) and lab grade water (4.4 ml) was treated with an aqueous solution of sodium periodate (99% purity, 3.13 g, 14.6 mmol (1.2 mole equivs)) in hydrochloric acid (12 molar, 1.46 ml 17.5 mmol (1.43 equiv)) and water (8.5 ml) over a period of 30 minutes keeping the temperature in the range 25-30° C. The suspension was stirred at ambient for 2 hours. HPLC analysis at near 2 hours showed 98.4 area % title compound present in the reaction mixture. Water (65 ml) was added dropwise over a period of 20 minutes. After complete addition, the mixture was cooled to 10° C., was stirred at 20° C. for 2 hours and the product filtered off. The bed was washed with water (2×15 ml) and then dried at 60° C. (house vacuum) for 18 hours leaving the title compound as a white granular solid (4.65 g, 96.3% th).

### EXAMPLE 1D

6α, 9α-difluoro-11β-hydroxy-16α-methyl-3-oxo-17α-hydroxy-androsta-1,4-diene-17β-carboxylic acid

A suspension of flumethasone (1 weight) in tetrahydrofuran (4.4 vol) and water (0.9 vol) was stirred at 22±3° C. until a clear solution was achieved. An aqueous solution of periodic acid (50% w/w, 1.33 weights, 1.2 equivalents) was added over approximately 45 minutes at a rate sufficient to maintain a reaction temperature of 25±5° C. A further portion of water (0.1 vol) was added as a line wash and the mixture was stirred at 22±3° C. for 2 hours (note, the hydroxyacid product starts to crystallise after approximately 1 hour of this stir period). Water (14 vol) was added to the suspension over at least 30 minutes maintaining a reaction temperature in the range 22±3° C. The mixture was cooled to approximately 10° C. and stirred for at least 1 hour at this temperature. The solid was filtered off and the bed washed with water (2×3 vol) at 22±5° C. The product was dried under vacuum at 60±5° C. to afford hydroxyacid as a white granular solid (expected yield 97% th).

### EXAMPLE 2

6α, 9α-difluoro-11β-hydroxy-16α-methyl-17α-propionyloxy-3-oxoandrosta-1,4-diene-17β-carbothioic acid, S-fluoromethyl ester

The compound of Example 2 may be prepared from Intermediate 2 following the processes described in GB Patent No. 2288877B.

### EXAMPLE 3

6α, 9α-difluoro-11β-hydroxy-16α-methyl-3-oxo-17α-propionyloxy-androsta-1,4-diene-17β-carbothioic acid S-(2-oxo-tetrahydro-furan-3-yl) ester

A mixture of Intermediate 2 (1 wt), Intermediate 3, and DMF (3.5 vol) is treated with 2,4,6-collidine (0.268 wt, 1.04 eq). The resulting solution is heated at 39-43° C. for approximately 4 h until complete by HPLC. 2M Hydrochloric acid (0.2 vol) is added and residual ethyl acetate is removed by vacuum distillation. The solution is warmed to 60° C. and water (approximately 2 vol) is added over 5-30 min at 60-65° C., seeding when a fine suspension has formed. The suspension is aged at 55-65° C. for at least 5 min and water (approximately 8 vol; total water added 10 vol) is added slowly at 49-60° C. The suspension is allowed to cool to room temperature and is aged for at least 30 min (typically overnight). The title compound is filtered off, washed with water (3×2 vol), and pulled dry.

The title compound is then purified using isopropanol recrystallisation, which comprises heating to reflux a suspension of the title compound in isopropanol (13.4 vol) and holding at reflux for at least 5 minutes. (At this stage the reaction mixture may be given a hot filtration). The solution is heated and maintained above 60° C. whilst filtered, purified water (5.6 vol) is added dropwise over at least 10 minutes. The suspension is cooled to 0-10° C. and then aged at <10° for at least 30 minutes. The solid is collected by vacuum filtration, washed with filtered purified water (2×3.4 vol) and dried under vacuum using a filter bed for at least 15 minutes. The product is dried in vacuo at up to 70° C. overnight. Expected yield: 99% w/w, 84% theory (uncorrected) from Intermediate 2.

The merits of the present invention may be seen by reference to the following Comparative Example:

### COMPARATIVE EXAMPLE

Example was prepared following a procedure analogous to that described in J. Med. Chem. (1994), 37, 3717, example 2b, (half scale), which describes conversion of des-16 alpha methyl dexamethasone to corresponding hydroxyacid via a cold isolation.

A suspension of flumethasone (5.42 g, 13.2 mmol) in tetrahydrofuran (27.5 ml) and lab grade water (3 ml) was treated with an solution of periodic acid (99% purity, 4.5 g, 19.8 mmol (1.5 mole equivs)) in lab grade water (45 ml) over a period of 15 minutes keeping the temperature in the range 20-30° C. The suspension was stirred at ambient for 2 hours and an essentially clear solution resulted. HPLC analysis at near 2 hours showed 98.9 area % title compound present in the reaction mixture. The reaction mixture was then added to a stirred mixture of water (75 ml) and crushed ice (125 g) over 5 minutes. The suspension was then stirred at 0-2° C. for 10 minutes and the solid was filtered off to give a very volumous gel (ca 100 ml).

This comparative example resulted in a voluminous product with a high level of associated solvent and it was not possible to remove this solvent by conventional solvent removal techniques, such as low temperature filtration. By contrast, in Example 1A, 1B, 1C. and 1D, the title compound was obtained in a relatively low volume (ca 4 ml) as a granular solid in a form which was readily filterable. In Example 1 which was performed on a larger scale the same low volume solid was obtained (ca 32 ml).

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

The above mentioned patents and patent applications are herein incorporated by reference.

1. A process for the preparation of a compound of formula (I) which comprises the following step: 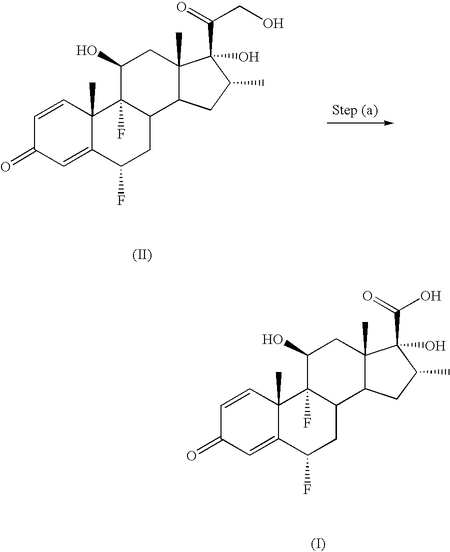
wherein step (a) comprises oxidation of a solution containing the compound of formula (II).

2. A process for the preparation of a compound of formula (A): 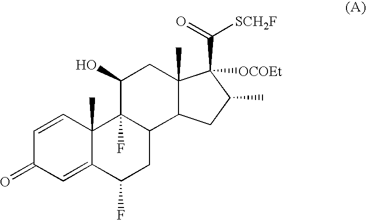
which comprises the following step: 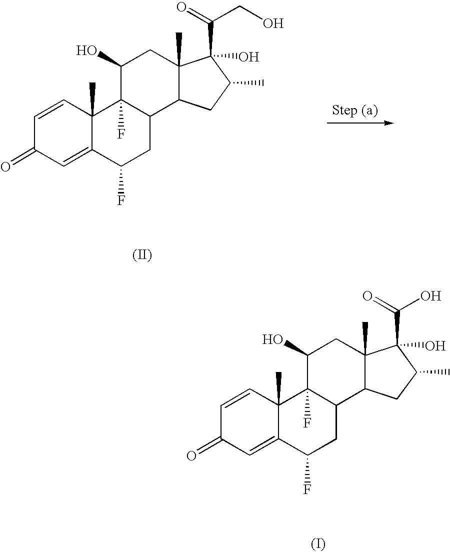
wherein step (a) comprises oxidation of a solution containing the compound of formula (II).

3. A process for the preparation of a compound of formula (A) according to claim 2 which comprises the following steps: 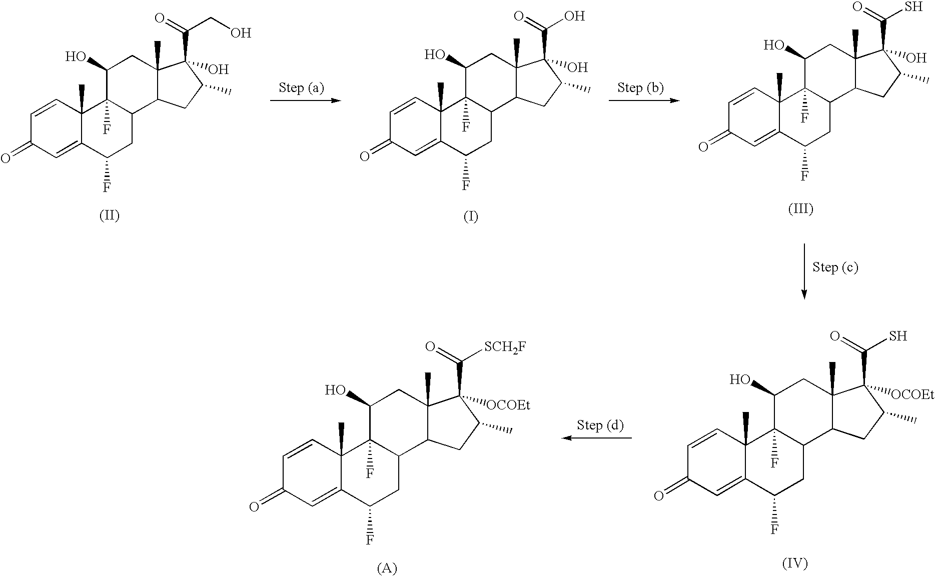
wherein step (a) comprises oxidation of a solution containing the compound of formula (II).

4. A process for the preparation of a compound of formula (B): 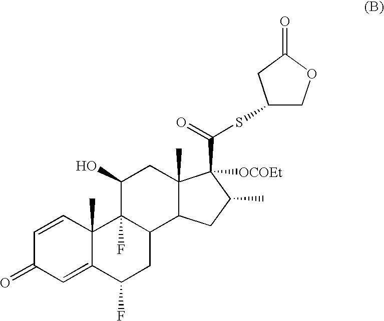
which comprises the following step: 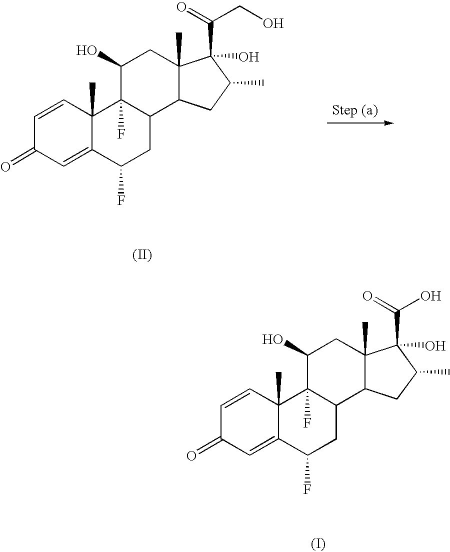
wherein step (a) comprises oxidation of a solution containing the compound of formula (II).

5. A process for the preparation of a compound of formula (B) according to claim 4 which comprises the following steps: 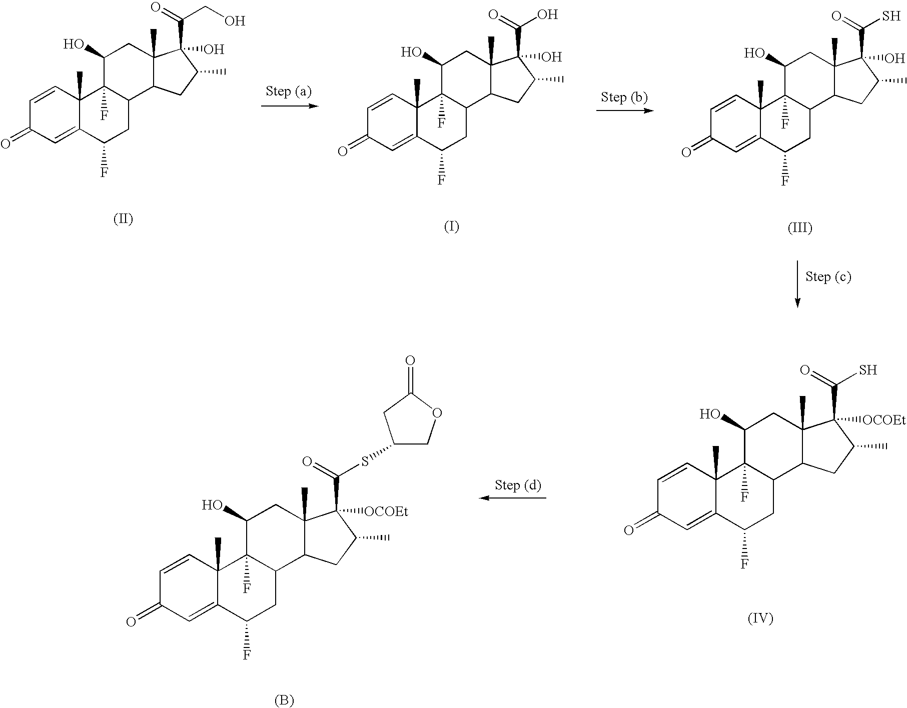
wherein step (a) comprises oxidation of a solution containing the compound of formula (II).

6. A process according to any one of claims 1 to 5 wherein step (a) is performed in the presence of a solvent comprising methanol, water, tetrahydrofuran, dioxan or diethylene glygol dimethylether.

7. A process according to claim 6 wherein step (a) is performed in the presence of methanol, water or tetrahydrofuran as solvent.

8. A process according to claim 6 wherein step (a) is performed in the presence of water and tetrahydrofuran as solvent.

9. A process according to any one of claims 1 to 5 wherein the solution containing the compound of formula (II) is cooled prior to oxidation eg. to a temperature less than approximately 10° C.

10. A process according to any one of claims 1 to 9 wherein the oxidation step comprises the use of a chemical oxidising agent.

11. A process according to claim 10 wherein the oxidising agent is periodic acid or iodic acid or a salt thereof.

12. A process according to claim 11 wherein the oxidising agent is periodic acid or sodium periodate.

13. A process according to claim 12 wherein the oxidising agent is periodic acid.

14. A process according to any one of claims 1 to 5 wherein the oxidation step comprises an oxidation reaction which utilises air and/or oxygen.

15. A process according to claim 14 wherein the solvent used in said reaction is methanol.

16. A process according to any one of claims 1 to 5 wherein step (a) is incubated at room temperature.

17. A process according to any one of claims 1 to 16 which further comprises a step (b) of precipitation of the compound of formula (I) in crystalline form from the reaction mixture by addition of an anti-solvent under temperature conditions of around 10° C. or higher.

18. A process according to claim 17 wherein the anti-solvent is water.

19. A process according to claim 17 or claim 18 wherein the temperature of step (b) is around ambient temperature or higher.

20. Compound of formula (I) in the form of a granular solid which is readily filterable obtainable by a process comprising:
(a) oxidising of a solution of a compound of formula (II) in water/tetrahydrofuran wth periodic acid in water at a temperature of around 20-30° C.; followed by
(b) precipitating the compound of formula (I) by addition of water at a temperature of around 22° C.

21. Use of a compound of formula (II): 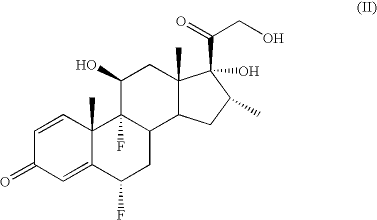
in the preparation of a compound of formula (I):

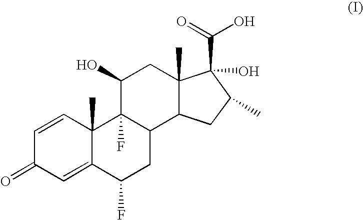

22. Use of a compound of formula (II): 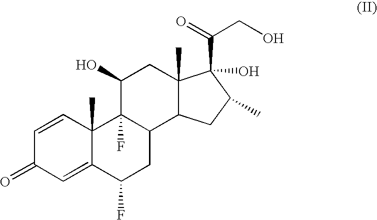
in the preparation of a compound of formula (A):

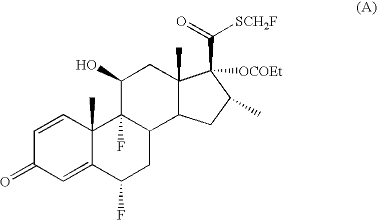

23. Use of a compound of formula (II): 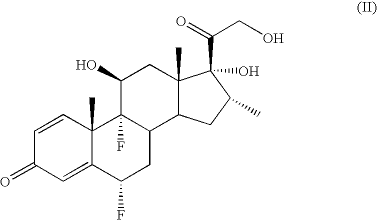
in the preparation of a compound of formula (B): [chemical structure]
